(19) United States
(12) Patent Application Publication
(10) Pub. No.: US 2009/0186370 A1
(43) Pub. Date: Jul. 23, 2009

(54) DIAGNOSTIC BIOMARKERS FOR VASCULAR ANEURYSM

(76) Inventors: Matthew F. Ogle, Fitchburg, WI (US); Jason C. Isenburg, Victoria, MN (US)

Correspondence Address: Dardi & Associates, PLLC;US Bank Plaza, Suite 2000, 220 South 6th Street, Minneapolis, MN, 55402, US

(21) Appl. No.: 12/355,384

(22) Filed: Jan. 16, 2009

Related U.S. Application Data
(60) Provisional application No. 61/011,648, filed on Jan. 18, 2008.

(51) Int. Cl. C12Q 1/37; G01N 33/00; C12Q 1/02

(52) U.S. Cl. 435/23; 436/111; 435/29

(57) ABSTRACT

Biomarkers for diagnosis and monitoring of vascular aneurysms are described in the context of the use of assays to measure a plurality of these biomarkers. Tissue degeneration, particularly elastin and/or collagen degradation, can be monitored within patient blood (serum) and/or urine to diagnose the presence, the progression, or the likelihood of rupture of aneurismal disease. Additionally, enzymes responsible for this degradation and other biomarkers responsible for the activation or inhibition of these enzymes can be monitored additionally or alternatively. Prompt diagnosis can provide the opportunity for intervention and potentially increase the health of patients by tempering the development of debilitating and life-threatening vascular aneurysms.

### CROSS REFERENCE TO RELATED APPLICATIONS

This application claims priority to U.S. provisional patent application Ser. No. 61/011,648, filed on Jan. 18, 2008 to Ogle et al., entitled “Diagnostic Biomarkers for Vascular Aneurysm,” incorporated herein by reference.

### FIELD OF THE INVENTION

The inventions, in general, are related to the detection and monitoring of vascular aneurysm using assays for biomarkers in biological fluids. The invention further relates to products that support biomarker assays for aneurysms.

### BACKGROUND

Aneurysms are degenerative diseases characterized by destruction of arterial architecture and subsequent dilatation of the blood vessel that may eventually lead to fatal ruptures. Some common locations for aneurysms include the abdominal aorta (abdominal aortic aneurysm, AAA), thoracic aorta, and brain arteries. In addition, peripheral aneurysms of the leg, namely the popliteal and femoral arteries are prevalent locations of this vascular pathology. The occurrence of such peripheral aneurysms appears to be strongly associated with the presence of aneurysms in other locations, as it has been estimated that 30 to 60% of peripheral aneurysm patients also have an AAA (Mousa A Y, Beauford R B, Henderson P, Patel P, Faries P L, Flores L, Fogler R. “Update on the diagnosis and management of popliteal aneurysm and literature review.” Vascular 2006;14(2):103-108; Watelet J. “Popliteal aneurysms.” J Cardiovasc Surg (Torino) 2007;48(3):263-265, both of which are incorporated herein by reference).

Aneurysms grow over a period of years and pose great risks to health. Aneurysms have the potential to dissect or rupture, causing massive bleeding, stroke, and hemorrhagic shock, which can be fatal in more than 80% of cases (Isselbacher E M. “Thoracic and abdominal aortic aneurysms.” Circulation 2005;111(6):816-828, incorporated herein by reference). AAAs are a serious health concern, specifically for the aging population, being among the top ten causes of death for patients older than 50. The estimated incidence for abdominal aortic aneurysm is about 50 in every 100,000 persons per year. Approximately 60,000 operations are performed each year in the U.S. for AAAs alone. In children, AAAs can result from blunt abdominal injury or from Marfan's syndrome, a defect in elastic fiber formation in walls of major arteries, such as the aorta.

### SUMMARY OF THE INVENTION

Diagnostic biomarkers for vascular aneurysms are described herein including, for example, desmosine and isodesmosine, peptide products of elastin degradation, products of collagen degradation, tissue plasminogen activator (t-PA), urokinase plasminogen activator (u-PA), matrix metalloproteinases such as MMP-2 and MMP-9, homocysteine, tissue inhibitors of metalloproteinases (TIMPs), a combination thereof or a ratio thereof. The diagnostic biomarkers can be obtained from patient blood, patient urine, or a combination thereof. In one embodiment, the amount of a biomarker, a combination of biomarkers, or a ratio between a plurality of biomarkers detected in patient blood or urine sample can be correlated to the onset and progression of an aneurysm condition.

In a first aspect, the invention pertains to a method for diagnosing an aneurysm, an aneurysm related disorder or an increased propensity thereof, estimating the stage of an existing aneurysm, or determining the propensity of possible future aneurysm rupture in a patient. The method generally comprises the steps of correlating levels of a plurality biomarkers measured within a biological sample obtained from a patient with a prescribed formula to evaluate the likelihood of aneurysm, the stage of an exiting aneurysm, or the likelihood of aneurysm rupture. The plurality of biomarkers can comprise at least an elastin degradation product and a collagen degradation product. In one embodiment, the elastin degradation product is desmosine, isodesmosine, or combination thereof. In one embodiment, the collagen degradation product is pyridinoline, deoxypyridinoline, or a combination thereof.

In some embodiments, the formula comprises the comparison of a level of the biomarkers against a reference level of the biomarkers, wherein the reference levels have been obtained through the measurement of the level of the plurality of biomarkers detected from healthy individuals or control patients at predetermined stage of aneurysm. In another embodiment, the formula comprises the comparison of a level of the biomarkers against a reference level of the biomarkers. The reference levels can be obtained based on previously obtained values from the patient. In one embodiment, the correlation of the plurality of biomarker levels is repeated in an interval from about six months to about 5 years, and at least one repeated biomarker level can be compared with the previously obtained value from the patient. In some embodiments, the interval can be from about 10 months to about 18 months. In further embodiments, the plurality of biomarkers further comprises matrix metalloproteinase. In particular, the matrix metalloproteinase can comprise matrix metalloproteinase 1, 2, 8, 9, 12, 13, 18, or a combination thereof. In one embodiment, the elastin degradation product comprises desmosine, isodesmosine, or a combination thereof, the collagen degradation product comprises pyridinoline, deoxypyridinoline, or a combination thereof, and the matrix metalloproteinase comprises matrix metalloproteinase 1, 2, 8, 9, 12, 13, 18, or a combination thereof. In some embodiments, the plurality of biomarkers can further comprise elastin degradation peptide, procollagen-IIIN-terminal propeptides, N-telopeptides of type I collagen, tissue inhibitors of metalloproteinases, tissue plasminogen activator, urokinase plasminogen activator, homocysteine or a combination thereof.

In another aspect, the invention pertains to another method for diagnosing an aneurysm, aneurysm related disorder or an increased propensity thereof, estimating the stage of an existing aneurysm, or determining the propensity of possible future aneurysm rupture in a patient. The method comprises the steps of correlating levels of a plurality of biomarkers measured within a biological sample obtained from a patient with a prescribed formula to evaluate the likelihood of aneurysm, the stage of an existing aneurysm, or the likelihood of aneurysm rupture. The plurality of biomarkers comprises at least desmosine and/or isodesmosine, and a metalloproteinase. In some embodiments, the matrix metalloproteinase comprises matrix metalloproteinase 1, 2, 8, 9, 12, 13, 18, or a combination thereof. In particular, the matrix metalloproteinase can comprise matrix metalloproteinase 2, 9, or a combination thereof. In one embodiment, the biomarkers assay can be repeated in an interval from about six months to about 5 years for a patient.

In yet another aspect, the invention pertains to a kit for performing a test to diagnose an aneurysm, an aneurysm related disorder or an increased propensity thereof, estimate the stage of an existing aneurysm, or determine the propensity of possible future aneurysm rupture in a patient. The kit can comprise one or more agents for detecting the level of each of a plurality of biomarkers in a biological sample from a patient to determine the levels of the plurality of biomarkers in the sample. The biomarkers are selected such that a correlation of the levels of the biomarkers with respect to known distributions of biomarker levels in a population can be used as an estimate of the likelihood of aneurysm, the stage of an existing aneurysm, or the likelihood of aneurysm rupture in the patient. In one embodiment, at least one agent can be used for the evaluation of the level of desmosine and isodesmosine in the sample.

### BRIEF DESCRIPTION OF THE DRAWINGS

FIG. 1 shows the correlation of urinary desmosine with aneurysm development

### DETAILED DESCRIPTION OF PREFERRED EMBODIMENTS

The approaches described herein provide a mechanism for efficient screening procedures for patients to identify individuals that should receive more extensive or costly screening for aneurysms due to the identification of relevant biological markers. The identification and selection of the biomarkers is based on the biochemical processes that can be associated with the formation and progression of aneurysms. In some embodiments, a plurality of biomarkers can be examined to provide an aggregate scope that can be tracked. The evaluation of a plurality of biomarkers may provide in some cases a more accurate assessment of the clinical development of the aneurysm, and in particular the review of a plurality of biomarkers can reduce the risk of false positive measurements. The biomarker assays can be used to identify evidence of possible aneurysm development, to track the progress of the disease, as well as to estimate the likelihood of aneurysm rupture. The availability of a low cost early detection procedure provides for intervention at an earlier stage of the disease. In the case of late stage aneurysm, the risk of aneurysm rupture may prompt the need of surgical intervention. The biomarker assays can be used in combination with other imaging or assay method to further determine the likelihood of aneurysm rupture.

Aneurysms may be caused by one or more of a variety of mechanisms including atherosclerotic disease, defects in arterial components, genetic susceptibilities, high blood pressure, and others. In many cases, aneurysms are associated with development of calcified lipid deposits or atherosclerotic plaques within the structure of the aorta. During plaque development, inflammation chronically occurs and the structure of the blood vessel continuously deteriorates. Further description of blood vessel deterioration can be found in Daugherty A, Cassis L A. “Mouse models of abdominal aortic aneurysms.” Arterioscler Thromb Vasc Biol 2004;24(3):429-434, incorporated herein by reference. The onset and progression of aneurysms are associated with enzymatic degradation of elastin and collagen etc. by matrix metalloproteinases (MMPs), which in turn are derived from activated vascular cells and infiltrating inflammatory cells. As a result, a characteristic of aneurysms is degeneration of arterial structural proteins including elastin and collagen, inflammatory infiltrates, calcification, and overall destruction of arterial architecture. This results in loss of mechanical properties of at least a section of a vessel and progressive dilatation.

Methods for diagnosing and identifying the degree of aneurysm expansion are available due to developments in high resolution imaging technology, such as CT scans, MRI imaging, or the like. Due to high cost for these procedures, high resolution imaging is not performed routinely on patients who are not exhibiting symptoms so that aneurysms may not be detected until they are at a more advanced stage. After initial diagnosis of a small AAA (larger than 2 cm in diameter), the most common medical approach is to periodically monitor its development (for instance, every 6 months) and if it reaches a certain stage (typically larger than 5.5 cm diameter), to apply surgical treatment. Similar monitoring can be applied to other aneurysms.

The current available diagnostic tools of aneurysm therefore largely depend on the size of aneurysm, which may or may not correlate well with the ultimate severity of the aneurysm. For instance, an aneurysm reached a 5.5 cm size may have entered a relatively stabilized stage of development and therefore is not prone to rupture or even further aneurysm development, in which case, an immediate surgically intervention may not be needed. The more accurate assessment of the aneurysm can be advantageous, especially considering that many aneurysm patients already have other diseases that could make a surgical intervention of the aneurysm risky. On the other hand, an aneurysm that is under the 5.5 cm cut-off may become unexpectedly latent and rupture. It is therefore advantageous to have alternative diagnostic method to estimate the further development of the aneurysm. The biomarker assays disclosed herein present such an alternative. When combined with the traditional size estimate, the biomarker assay could provide more accurate assessment of the condition of the aneurysm and consequently, the choice and timing of intervention. In particular, the biomarker assays provide measurements of active in vivo mechanisms that are involved in aneurysm development.

Appropriate surgical treatment can involve endovascular stent graft repair (placement of a tube inside the vessel) or complete replacement of the diseased aorta with an artificial mesh vascular graft. Surgical treatment of aneurysms saves thousands of lives every year and improves quality of life. However, survival rates can drop to only 50% at 10 years post-operative due to surgery-related complications or device-related problems. Moreover, open surgery for full-size graft insertion is highly invasive, limiting its use to those patients with high operative risk. In addition, endovascular stents are anatomically appropriate for only 30% to 60% of AAA patients at the outset and present the risk of endoleaks and graft displacement. Issues relating to stent and surgical treatments of AAA are discussed further in Isselbacher, “Thoracic and abdominal aortic aneurysms,” Circulation 2005;111(6):816-828, incorporated herein by reference.

Early diagnosis of aneurysms could provide the opportunity for a new class of treatments and interventions for these potentially debilitating and life-threatening vascular pathologies. By doing so, treatment could be offered earlier, which may be advantageous since age is one of the major risk factors associated with the current approaches to treat aneurysms. See, for example, Irvine et al., “A comparison of the mortality rate after elective repair of aortic aneurysms detected either by screening or incidentally,” Eur J Vasc Endovasc Surg 2000;20(4):374-378, incorporated herein by reference. Approaches and related device and delivery systems for non-surgical treatment of aneurysms are described in published U.S. Patent Application 2006/0240066 to Vyavahare et al., entitled “Elastin Stabilization of Connective Tissue,” U.S. Patent Provisional Application 61/113,881 to Isenburg et al, entitled “Compositions for Tissue Stabilization,” U.S. Patent Provisional Application 61/066,688 to Isenburg et al, entitled “Treatment of Aneurysm with Application of Elastin Stabilization Agent Embedded in a Delivery System,” and U.S. patent application Ser. No. 12/173,726 to Ogle et al, entitled “Devices for the Treatment of Vascular Aneurysm,” all of which incorporated herein by reference.

In addition, assessment tools which might assist in predicting the status of aneurysm progression would be highly beneficial. For instance, it is estimated that only half of all patients who have undergone surgical bypass or replacement for an AAA would have eventually suffered an aneurismal rupture if ignored. See, for example, Lindholt J S. “Activators of plasminogen and the progression of small abdominal aortic aneurysms.” Ann N Y Acad Sci 2006;1085:139-150, incorporated herein by reference. More advanced methods of monitoring aneurysm progression, such as assay panels with combinations of the biomarkers proposed herein, may provide a clearer predictive method of future rupture. By clarifying which patients are at highest risk for future rupture, treatments and interventions can be carried out accordingly.

In general, some biomarkers track elastin breakdown, other biomarkers track collagen breakdown, additional biomarkers track enzymes that are responsible for the structural protein breakdown, and other biomarkers are responsible for the regulatory enzymes of the degradation processes. Desmosine is a modified amino acid specific to elastin; its systemic release indicates elastin breakdown, and this biomarker can be useful by itself for monitoring aneurysm progression. However, in some embodiments it is desirable to track a set of biomarkers with at least one biomarker correlated with elastin breakdown and at least one biomarker correlated with collagen breakdown. Optionally, the set of biomarkers can further include at least one biomarker that is an enzyme that is responsible for the structural protein breakdown and/or other biomarkers that are responsible for the regulation of the proteolytic enzyme. As described further below, the tracking of multiple biomarkers can be used to determine a score that can be correlated with an evaluation of the stage of the disease. The use of a plurality of biomarkers can provide a significantly more accurate assessment of possible damage to vascular tissue, which correlates with aneurysm development and progress.

Antibodies and/or specific binding agents relating to a specific biomarker can be used to develop assay for the biomarker. In some embodiments, the apparatus performs simultaneous or sequential assays of a plurality of biomarkers, for example, using a single test device. The assays and instructions of how to perform the assays for the plurality of biomarkers can be offered in a kit to facilitate the process of detecting one or more biomarker of interest. Treatment regimen can be coordinated with continued monitoring of existing aneurysm at time intervals, which can be selected in accordance to the condition of the aneurysm.

The processing of a plurality of biomarkers for aneurysms can provide a more accurate assessment of the status for the disease. This more accurate assessment is consistent with the ability to perform earlier diagnosis as well as improved monitoring of the course of disease using the analysis of biological fluids. The improved diagnosis and monitoring provides for earlier treatment of the disease as well as the possibility of lower cost monitoring with reduced use of expensive imaging. The biomarker assay could additionally compliment the existing imaging methods to more accurately assess the likelihood of aneurysm rupture and the interventional method that could be used.

### Desmosine, Isodesmosine and Product of Elastin Degradation

Fibrillar proteins elastin and collagen (types I and III) are the principal structural proteins of the aorta and other arteries, which impart both strength and resilience to the vessel wall. Elastin in particular endows vascular tissue with the ability to extend and recoil repetitively. Elastin is primarily composed of the amino acids glycine, valine, alanine, and proline. It is a specialized protein with a molecular weight of 64 to 66 kDa, and an irregular or random coil conformation made up of 830 amino acids. Elastin is made by linking many soluble tropoelastin protein molecules, in a reaction catalyzed by lysyl oxidase, to make a massive insoluble, durable cross-linked array. Due to its insolubility and extremely long biological half-life, elastin is generally perceived to be resistant to degradation. However, there are a specific set of enzymes, matrix metalloproteinases (particularly MMP-2, MMP-9, and MMP-12), which are capable of degrading elastin.

There are consistent reports of severe elastin degradation within aneurysmal tissues, as evidenced by heavy degeneration of the arterial architecture, decreased medial elastin content, and disrupted or fragmented elastic lamellae. This degradation is particularly significant when one considers the inability of elastin to promptly revitalize itself (as evidenced by its nearly 70-year biological half-life), unlike some other relatively dynamic matrix components. Furthermore, degradation of elastin results in the release of soluble elastin peptides as well as the desmosine and isodesmosine cross linkers. The degraded elastin peptides are not passive products of the degradation process; rather, it has been demonstrated that the degraded elastin peptides are active in protease production, chemotaxis, cellular proliferation, and various other biological activities. The release of elastin peptides can result in a cascade of even more matrix degradation. In particular, it has been shown that interactions between these peptides and smooth muscle cells increase expression of the elastin laminin receptor (ELR). This binding with ELR, a 67 kDa receptor found on a number of cell types, subsequently results in the promotion of greater MMP synthesis both at the mRNA and protein levels.

Numerous studies have confirmed this correlation between upregulated MMP activity and the presence of elastin peptides. The use of luminally-perfused elastin peptides as an aneurysm animal model, which elicits elevated MMP levels and matrix degradation at the site of perfusion, also demonstrates the biological activity of these peptides. The demonstrated bioactivity of elastin peptides underscores the clinical significance of elastin degradation within aneurysmal tissues and the subsequent possibility to use elastin degradation product as biomarkers for diagnosis of aneurysm and/or monitoring the progression thereof.

Desmosine and isodesmosine are integral components of elastin, formed from four side chains of lysine of soluble tropoelastin protein and constituting cross linkages in the insoluble elastin. The structure of desmosine is shown in Formula I, with a pyridinium core having four amino carboxyl side chains at 1, 3, 4, and 5 position of the pyridinium core. Isodesmosine is a structural isomer of desmosine, with four amino carboxyl side chains at 1, 2, 3, and 5 position of the pyridinium core.

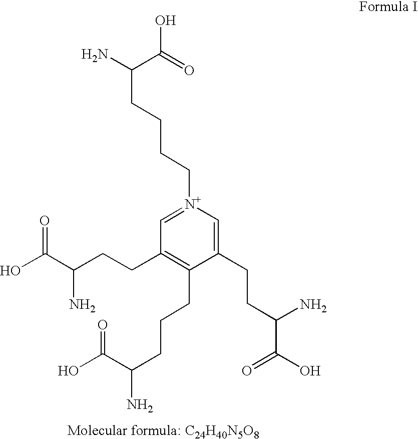

Desmosine and isodesmosine are amino acid derivatives that are specific to elastin. When elastin goes through degradation, desmosine and isodesmosine are released as the elastin degradation products. Additionally, desmosine and isodesmosine are not metabolized and are passed to urine for removal from the body. Because the onset and progression of aneurysms are associated with enzymatic degradation of elastin, detection of a predetermined level of desmosine and isodesmosine in patient blood or urine can be an indication of aneurysm. Furthermore, increasing level of desmosine and isodesmosine in patient blood or urine sample relative to corresponding control values from healthy individuals can be correlated to the progression of aneurysm. Desmosine and isodesmosine therefore, can be used as diagnostic biomarkers for vascular aneurysm.

There are several methods for measuring desmosine, including, for example, enzyme-linked immunosorbent assay (see, e.g., Osakabe T. et al. “Comparison of ELISA and HPLC for the determination of desmosine and isodesmosine in aortic tissue elastin”, J. Clin Lab Anal Vol. 9 pgs 293-296 (1995)); isotope dilution (see, e.g., Stone P. J. et al. “Measurement of urinary desmosine by isotope dilution and high performance liquid chromatography”, Am Rev Respir Dis Vol. 144 pgs 284-290 (1991)); high performance liquid chromatography (see, e.g., Covault H. P. et al. “Liquid-chromatographic measurement of elastin”, Clin Chem Vol. 28 pgs 1465-1468 (1982)); and/or radioimmunoassay (see, e.g., Starcher B. “A role for neutrophil elastase in the progression of solar elastosis”, Connect Tissue Res Vol. 31 pgs 133-140 (1995)), with each of these four references being incorporated herein by reference. Desmosine and isodesmosine may be also quantified by procedures involving ultraviolet spectroscopy, fluorescence spectroscopy, mass spectroscopy, or other spectroscopic techniques well-known to the art. The measurement of desmosine in bodily fluids is described in U.S. Pat. No. 5,354,662 to Stone et al., entitled “Measuring Tissue Breakdown Products in Bodily Fluids,” incorporated herein by reference. U.S. Pat. No. 7,166,437 to Cantor et al., entitled “Measurement of Elastic Fiber Breakdown Products in Sputum,” reported measurement of desmosine and isodesmosine using paper chromatography, which is incorporated herein by reference. Moreover, assay of elastin peptide and desmosine in urine are found to be useful in characterizing elastin degradation in a patient with aneurysm, see, Osakabe et al., “Characteristic change of urinary elastin peptides and desmosine in aortic aneurysm,” Biol. Pharm. Bull. 1999;22(8): 854-857, incorporated herein by reference.

Other direct products of elastin degradation, such as elastin peptides, can be used as biomarkers as well. Hageman proposed using immunological method to detect elastin degradation product such as the peptide Val-Gly-Val-Ala-Pro-Gly (VGVAPG) in U.S. Pat. No. 7,108,982 entitled “Diagnostics and the Therapeutics for Macular Degeneration”, incorporated herein by reference. In one embodiment, the amount of desmosine and isodesmosine detected in patient blood or urine sample can be directly correlated to the onset and progression of aneurysm. In another embodiment, the amount of elastin degradation product detected in patient blood or urine sample can be directly correlated to the onset and progression of aneurysm. In one embodiment, the elastin degradation product is VAVAPG peptide.

### Product of Collagen Metabolism

Additional useful biomarkers for aneurysm evaluation are related to collagen metabolism. Similarly to elastin degradation, the onset and progression of aneurysms are associated with abnormal metabolism of collagen in connective tissue. See, for example, Loftus I M, Thompson M M. Vasc Med 2002; 7(2): 117-133, incorporated herein by reference. In the course of aneurysm development, it has been suggested that the processes of degradation and regeneration of collagen alternates. Once the collagen degradation reaches a particular degree, the rupture of the aneurysm tissue may occur. See, for example, Choke E, Cockerill G, Wilson W R, et al. Eur J Vasc Endovasc Surg 2005; 30(3): 227-244, incorporated herein by reference. Detection of an abnormal level of collagen metabolite in patient blood or urine can be an indication of aneurysm. Abnormal level of collagen metabolites in patient blood or urine sample can be correlated to the progression of aneurysm. Collagen metabolites therefore, can be used as diagnostic biomarkers for vascular aneurysm. In one embodiment, the amount of collagen metabolite detected in patient blood or urine sample can be correlated to the onset and progression of aneurysm.

For example, an increase in collagen turnover can be determined by the concentration of the amino terminal propeptide of type III procollagen in patient fluids. Procollagen-IIIN-terminal propeptides is one of the major collagen degradation and turnover products. In one embodiment, the amount of procollagen-IIIN-terminal propeptides detected in patient blood or urine sample can be correlated to the onset and progression of aneurysm. The detection of the breakdown products of collagen III, which is a collagen type generally associated with artery walls, is described in the following patents, which are incorporated herein by reference: U.S. Pat. No. 5,679,583 to Brocks et al, entitled “Monoclonal Antibodies for the Selective Immunological Determination of Intact Procollagen Peptide (Type III) and Procollagen (Type III) in Body Fluids,” U.S. Pat. No. 6,010,862 to Eyre, entitled “Methods of Detecting Collagen Type III Degradation In Vivo,” and U.S. Pat. No. 6,210,902 to Bonde et al., “Estimation of the Fragmentation Pattern of Collagen in Body Fluids and the Diagnosis of Disorders Associated with the Metabolism of Collagen.”, incorporated herein by reference. Similarly, amino telo-peptides of type I collagen (NTx), a known degradation product of collage type I can be used as an aneurysm detection biomarker as well. A commercially available assay Osteomark™ from Ostex International can be used to analyze NTx in serum or urine sample from a patient.

Pyridinoline (PYR) is a 3-hydroxypyridinium cross-linking compound found in mature collagen. Deoxypyridinoline (dPYR) is an analog of PYR present in minor amount that also functions as a cross-linker in mature collagen. Mature collagen is stabilized mainly by these two stable cross-linkers. During collagen degradation at an aneurysm, PYR and dPYR are released from the aneurysm tissue either as a free molecule or bound to a fragment of collagen peptide. Once released, PYR and dPYR are not significantly further metabolized, and therefore these compounds can be used as specific biomarkers for collagen. For example PYR and dPYR in particular are used as a tool to assess bone resorption rates in healthy individuals and in patients with enhanced risk of developing metabolic bone disease. See, Miller et al, “Practical Clinical Application of Biochemical Markers of Bone Turnover: Consensus of an Expert Panel,” J Clin Densitom, 1999, 2(3):323-42, incorporated herein by reference. The PYR: dPYR ratio in bone is 3.5:1. In order to estimate the amount of PYR derived from soft tissue (stPYR), Stone et al proposed to deduct the PYR produced by bone resorption from the overall PYR detected using the formula: [stPYR]=[PYR]−3.5×[dPYR], as described in Stone et al., “Cross-linked Elastin and Collagen Degradation Products in the Urine of the Patients with Scleroderma,” Arthritis Rheum 1995; 38: 517-24, incorporated herein by reference. Using the formula proposed by Stone, Istok et al. was able to correlate stPYR successfully with fibrosis of skin. See, Istok et al., “Increased Urinary Pyridinoline Cross-link Compounds of Collagen in Patients with Systemic Sclerosis and Raynaud's Phenomenon,” Rheumatology 2001, 40:140-146, incorporated herein by reference.

Similarly, when using PYR and dPYR as biomarkers for aneurysm, the bone resorption factor can be considered. Studies suggest reduced overall collagen content in aneurysmal tissue accompanied by increased cross-links in old mature collagen. Due to an alteration of PYR and dPYR levels in aneurysm patients relative to levels in healthy patient, PYR and dPYR can be used as biomarkers for the progression of the disease. The changes in matrix proteins is described further in Carmo et al. “Alteration of Elastin, Collagen and their Cross-links in Abdominal Aortic Aneurysms”, Eur J Vasc Endovasc Surg 23, 543-549, 2002, incorporated herein by reference. Urinary and serum PYR and dPYR level can be measured using commercially available assays such as immunoassays provided by Quidel Corp. Alternatively PYR and dPYR may be quantified by procedures involving ultraviolet spectroscopy, fluorescence spectroscopy, mass spectroscopy, or other spectroscopic techniques well-known to the art.

### Matrix Metalloproteinases

The matrix metalloproteinase (MMP) family of enzymes is involved in the breakdown of extra cellular matrix (ECM) in normal physiological processes, such as embryonic development, reproduction, and tissue remodeling, as well as in disease processes, such as arthritis, atherosclerosis, and aneurysm. Most MMP's are secreted as inactive proproteins which are activated when cleaved by extracellular proteinases. Specific to aneurysms, the most widely investigated MMPs are those enzymes that have been identified as degrading elastin: MMP-2 (72 kDa gelatinase), MMP-9 (92 kDa gelatinase), and MMP-12 (macrophage metalloelastase). The role of such enzymes has been validated by experimental results as well as studies on explanted human AAAs.

It is believed that MMP-2 plays a significant role in initial aneurysm formation, as it has been found in high concentrations within small or early-stage aneurysms in experimental animal models and human tissue. See, for example, Ailawadi et al., “Current concepts in the pathogenesis of abdominal aortic aneurysm,” J Vasc Surg 2003;38(3):584-588, incorporated herein by reference. This trend of elevated MMP-2, which can be secreted by smooth muscle cells and fibroblasts, can be correlated with increased enzyme activity, as described in Freestone et al., “Inflammation and matrix metalloproteinases in the enlarging abdominal aortic aneurysm,” Arterioscler Thromb Vasc Biol 1995;15(8):1145-1151, incorporated herein by reference. MMP-2 additionally degrades type IV collagen, the major structural component of basement membranes and plays a role in regulation of vascularization and the inflammatory response.

MMP-9 has also been found to contribute to AAA development. MMP-9 has been observed to be capable of degrading elastin, but to a somewhat lesser degree than MMP-2. See, Senior et al., “Human 92- and 72-kilodalton type IV collagenases are elastases,” J Biol Chem 1991;266(12):7870-7875, incorporated herein by reference. MMP-9 has been measured in human aneurysmal tissue at levels 10 times greater than normal aorta. This same study revealed that the predominant source of MMP-9 overexpression was localized to macrophages, as described in Thompson et al., “Production and localization of 92-kilodalton gelatinase in abdominal aortic aneurysms. An elastolytic metalloproteinase expressed by aneurysm-infiltrating macrophages,” J Clin Invest 1995;96(1):318-326, incorporated herein by reference. MMP-9 is the most abundant elastin degrading enzyme found within aneurysmal tissues and possesses strong collagen degrading capabilities as well, as described in Dawson et al., “Pharmacotherapy of abdominal aortic aneurysms,” Curr Vasc Pharmacol 2006;4(2):129-149, incorporated herein by reference.

The involvement of MMP-2 and MMP-9 with respect to aneurysm formation have been further authenticated with the implementation of knockout mice models. MMP-2 and MMP-9 knockout mice were extremely resistant to experimental aneurysm formation, displaying virtually no increase in aortic diameter (less than 10%) in both cases, as described in Longo et al., “Matrix metalloproteinases 2 and 9 work in concert to produce aortic aneurysms,” J Clin Invest 2002;110(5):625-632, incorporated herein by reference. This study revealed that the absence of either gelatinase offered protection toward the tissue's matrix, thus preventing aneurysm formation. As a result, an interaction between these two MMPs is apparently required to properly elicit aneurysm formation; in other words, evidence suggests that the enzymes work synergistically within this pathology.

MMP-12, also known as macrophage metalloelastase, degrades soluble and insoluble elastin. MMP-12 has been found in human aneurysmal tissue, as described in Curci, “Expression and localization of macrophage elastase (matrix metalloproteinase-12) in abdominal aortic aneurysms,” J. Clin Invest 1998; 102:1900-10, incorporated herein by reference. The expression pattern of MMP-12 is distinct from other elastolytic enzymes and suggests its unique role in elastin degradation. MMP-12 knockout mice have been observed to display somewhat improved resistance to aneurysm formation in an experimental model. However, in the mouse models the effect of MMP-12 knockout was not nearly as significant as compared to MMP-2 and MMP-9 knockout studies.

Altered MMP-2, MMP-9, and MMP-12 activity in patients relative to normal control individuals can therefore be correlated to the onset and progression of aneurysm. Detection of an abnormal level of alteration of MMP-2, MMP-9, and MMP-12 in patient blood or urine can be an indication of aneurysm or can be correlated to the progression of aneurysm. MMP-2, MMP-9, and MMP-12 therefore, can be used as diagnostic biomarker(s) for vascular aneurysm.

Other MMPs that contribute to ECM degradation directly and indirectly can be used as biomarkers as well. Collagenases for example are the only known mammalian enzymes capable of degrading triple-helical fibrillar collagens. The collagenases include, for example, MMP-1, MMP-8, MMP-13, and MMP-18. MMP-1 is an interstitial collagenase that has been observed to be increased relative to normal levels in human aneurysms. In addition to possessing the ability to directly digest collagen, MMP-1 can also cleave latent MMP-9 into its active form. MMP-8, a relatively powerful neutrophil collagenase, has been observed to be elevated within human AAA tissue. This enzyme has also been associated with atherosclerosis and, more recently, has been shown to be in abundance along with MMP-9 at the site of aneurysm rupture. MMP-13, also referred to as collagenase-3, has also been observed to be overactive in aneurysmal aorta. This collagenase is localized to smooth muscle cells (SMCs) and is adept in activating other latent MMPs.

Altered MMP-1, MMP-8, MMP-13, and MMP-18 activity in patients relative to normal control patients can therefore be correlated to the onset and progression of aneurysm. Detection of an abnormal level of alteration of MMP-1, MMP-8, MMP-13, and MMP-18 in patient blood or urine can be an indication of aneurysm or can be correlated to the progression of aneurysm. MMP-1, MMP-8, MMP-13, and MMP-18 therefore, can be used as diagnostic biomarker(s) for vascular aneurysm individually and/or in combination with one or more additional biomarkers.

In one embodiment, the amount of MMP-1, MMP-8, MMP-2, MMP-9, MMP-10, MMP-13, and MMP-18, or a combination thereof detected in patient sample can be correlated to the onset and progression of aneurysm. The expression of MMPs in patients at different stages of aneurysm is likely to present its distinct pattern relative to normal control individuals. Accordingly, in another embodiment, the amount of MMPs that contribute to ECM degradation directly and indirectly detected in patient sample can be directly correlated to the onset and progression of aneurysm. The patient sample can be blood sample, urine sample or other bodily fluids.

Various methods have been developed for the detection of matrix metalloproteinases, including, for example, enzyme immunoassays using ELISA kits, such as from commercial sources, Zymography that identifies MMPs by degradation of their preferential substrate and by their molecular weight, and mass spectrometry using the concept of proteomics. Lopez-Avila et al. has proposed a dual separation method employ both immunoaffinity chromatography using an anti-MMP catalytic domain antibody to fish out the MMPs from tissue (i.e. human serum or plasma) and high-pressure liquid chromatography to separate the MMPs as individual proteins, see Lopez-Avila V. et al., “Methods for detection of matrix metalloproteinases as biomarkers in cardiovascular disease,” Clinical Medicine: Cardiology 2008:2 75-87, incorporated herein by reference.

### Tissue Inhibitors of Metalloproteinases (TIMPs)

Tissue inhibitors of matrix metalloproteinases (TIMPs) are a family of natural inhibitors that control the activity of MMPs in the ECM. Abnormal levels of the ratio between MMP and TIMP in patient blood or urine sample can be correlated to vascular pathologies and thus the progression of aneurysm. Detection of an abnormal ratio between MMP and TIMP in patient blood or urine can be used as an indication of aneurysm. The ratio between MMP and TIMP therefore, can be used as diagnostic biomarker(s) for vascular aneurysm. In one embodiment, the ratio between MMP and TIMP detected in patient blood or urine sample can be correlated to the onset and progression of aneurysm. The measurement of TIMP Type-l is described in U.S. Pat. No. 7,108,983 to Holten-Andersen et al., entitled “Tissue Inhibitor of Matrix Metalloproteinases Type-1 (TIMP-1) as a Cancer Marker,” incorporated herein by reference. TIMP Type-1 has been observed to form complexes with MMP-9.

Tissue Plasminogen Activator (t-PA)

Tissue plasminogen activator (abbreviated PLAT or tPA) is a serine protease normally found on the surface of endothelial cells of veins, capillaries, the pulmonary artery, heart, and uterus, and is secreted after vascular injury. It converts the proenzyme plasminogen to plasmin, a fibrinolytic enzyme that contributes to extracellular matrix (ECM) degradation directly and indirectly (by activating other enzymes). Altered tPA activity in patients relative to normal levels can be correlated to the onset and progression of aneurysm. Detection of abnormal level of tPA in patient blood or urine can be an indication of aneurysm. Altering level of tPA in patient blood or urine sample can be correlated to the progression of aneurysm. TPA therefore, can be used as a diagnostic biomarker for vascular aneurysm. Other fibrinolytic enzyme that contributes to ECM degradation directly and indirectly can be used as biomarkers as well. In one embodiment, the amount of tPA detected in patient blood or urine sample can be directly correlated to the onset and progression of aneurysm. In another embodiment, the amount of fibrinolytic enzyme that contributes to ECM degradation directly and indirectly detected in patient blood or urine sample can be correlated to the onset and progression of aneurysm. Assays for the measurement in biological samples of tPA and urokinase plasminogen activator is described in U.S. Pat. No. 7,045,280 to Elvin et al., entitled “Assay Method,” incorporated herein by reference.

Urokinase Plasminogen Activator (uPA)

Urokinase (Abbokinase), also called urokinase-type Plasminogen Activator (uPA), is a serine protease (EC 3.4.21.73). Urokinase was originally isolated from human urine, but is present in several physiological locations, such as blood stream and the ECM. The primary physiological substrate of uPA is plasminogen, which is an inactive zymogen form of the serine protease plasmin. Activation of plasmin triggers a proteolysis cascade, which depending on the physiological environment participate in thrombolysis or ECM degradation. Similarly, increased uPA activity can be correlated to the onset and progression of aneurysm. Detection of abnormal level of uPA in patient blood or urine is an indication of aneurysm. An altered level of uPA in patient blood or urine sample relative to normal levels can be correlated to the progression of aneurysm. UPA therefore, can be used as a diagnostic biomarker for vascular aneurysm. Other fibrinolytic enzyme that contributes to ECM degradation directly and indirectly can be used as biomarkers as well. In one embodiment, the amount of uPA detected in patient blood or urine sample can be correlated to the onset and progression of aneurysm.

### Homocysteine

Increased levels of the physiological amino acid homocysteine are considered a risk factor for vascular disease, which causes an intense remodeling of the ECM in arterial walls, particularly activation of metalloproteinases, tPA, and uPA. Increased levels of homocysteine therefore can be correlated to the onset and progression of aneurysm. Detection of abnormal level of homocysteine in patient blood or urine can be an indication of an aneurysm. Specifically, an elevated level of homocysteine in a patient's blood or urine sample relative to normal levels can be correlated to the progression of aneurysm. Homocysteine therefore, can be used as diagnostic biomarker(s) for vascular aneurysm. An assay for the measurement of homocysteine in biological samples is described further in U.S. Pat. No. 6,867,014 to Kawasaki et al., entitled “Enzymatic Cycling Assays for Homocysteine and Cystathionine,” incorporated herein by reference.

Other activators of metalloproteinases, tPA, and/or uPA that contribute to ECM degradation can be used as biomarkers as well. In one embodiment, the amount of homocysteine detected in patient blood or urine sample can be correlated to the onset and progression of aneurysm. In another embodiment, the amount of other activators of metalloproteinases, tPA, and/or uPA that contribute to ECM degradation detected in patient blood or urine sample can be directly correlated to the onset and progression of aneurysm.

### Combination of Biomarkers

While the detection of an amount of a single biomarker can be used as an indication of aneurysm and the progression of the disease, a combination of biomarkers can also be used effectively. In general, the measurement of a plurality of biomarkers can be used effectively for the performance of an evaluation of an aneurysm condition. In one embodiment, a combination of an elastin degradation product and a collagen degradation product is used to detect the onset and/or progression of an aneurysm. In additional or alternative embodiments, an enzyme that contributes to ECM degradation and/or other biomarkers responsible for the regulation of the enzyme can be used in addition or as substitutes for an elastin degradation product and/or a collagen degradation product. Furthermore, a combination of biomarkers from each group can be used in some embodiments. In some embodiments, the use of a plurality of biomarkers, especially of different types, can reduce the incidence of false negative conditions: since depending on the stage of the disease all of the biomarkers may not be simultaneously shifted from normal values.

For at least several of the biomarkers described herein, the level of the biomarker can be within a narrow range for healthy individuals. A cut off value can be reasonably selected that yields a reasonably low number of false positive readings. Thus, a value over the cut off can be indicative of an aneurysm or another connective tissue disease. However, due to the complexity aneurysms and other connective tissue disease as well as due to the changing conditions during the evolution of the disease, the levels of a particular biomarker may not be outside of the normal distribution even if significant physiological manifestations of the disease are taking place. Thus, the reliance on a single biomarker with a selected cut off value can result in an undesirable incomplete view of the status of the disease condition.

An evaluation of an aneurysm status can be developed as a multivariable function based on the input of the various biomarker measurements. Thus, a score S(b₁,b₂,b₃,b₄) can be a function of individual biomarker measurements, b₁,b₂,b₃,b₄. In general, the score can be determined from 2, 3, 4, or more biomarkers and can reach 10 or more biomarkers. As an example, one evaluation can involve the checking of a plurality of markers to see if the level of each marker is outside of a selected cut off value, which can be an upper or lower limit as appropriate. Through the evaluation of a plurality of markers, the likelihood of false negative readings is reduced. Also, more complex formulas can also be used based on the measurements of levels of a plurality of biomarkers.

In general, the score can be a more accurate determinant of the disease progression. The score can be effectively correlated with vessel health as a virtual histology without any direct imaging. In one embodiment, the elastin degradation product in the combination of biomarkers is desmosine. In other embodiments, the collagen degradation product in the combination of biomarkers is pyridinoline. In one embodiment, the enzyme in the combination of biomarkers is tPA, uPA, MMP-1, MMP-2, MMP-8, MMP-9, MMP-13, MMP-18 or a combination thereof. In other embodiments, other biomarker responsible for the regulation of the enzyme is homocysteine, TIMPs, or a combination thereof.

Because at the various stage of aneurysm, different biomarkers may be present at different altered levels relative to a normal state, it can be very advantageous to develop an evaluation methodology based on a combination of biomarkers to map out the stage of aneurysm. For example, elastin degradation is a prominent feature of aneurysm. The detection of increased level of elastin degradation product desmosine and isodesmosine therefore can be an indication of active aneurysm. Towards the end of the aneurysm development cycle, however, most of the elastin in the aneurysmal tissue has already been depleted, the elastin degradation product detected at this stage, therefore, is likely to be less than the value detected at the peak of aneurysm development. The advantages of using a combination of biomarkers therefore can be very significant.

The level of biomarkers in healthy individuals can be measured and recorded to provide reference values of the levels of biomarkers. Specifically, the recorded level of biomarker in healthy individuals forms a base line value that can be compared with those levels in patients that are suspected of aneurysm. Because the level of biomarkers in an individual may vary significantly during his or her life span, different base line values can be established for different age and gender groups.

Similarly, the levels of biomarkers in individuals at different stage of aneurysm can be measured and recorded to facilitate an evaluation of the evolution of the status of a particular patient over time. The biomarker levels from the aneurysm patients can be adjusted with the recorded base line level to establish alteration pattern of biomarkers at different stage of aneurysm. An established pattern of the aneurysm biomarkers that differ from values in healthy patients can in turn be used to screen patients. The biomarkers can be used as a part of a routine physical or can be used as a stand alone screen for demographics that are suspected of aneurysm. Once an altered level of biomarkers in a patient is detected, the pattern of biomarkers levels can be compared with the established patterns of aneurysm biomarkers from other aneurysm patients or with previously determined patterns of aneurysm biomarkers of the same patient to estimate the stage of the aneurysm. Appropriate interventional method can subsequently be employed to treat the patient. For patients that are in early stage of aneurysm, no interventional procedure may be used, and follow up screen tests can be carried out. In one embodiment, the patent is screened at intervals from about 10 month to about 18 months. In another embodiment, the patient is screened at intervals from about 6 months to 3 years. In yet another embodiment, the patients is screened at intervals from about 6 months to 5 years. A person of ordinary skill in the art will recognize that additional ranges of screen intervals within the explicit ranges above are contemplated and are within the present disclosure.

### Assay Measurement Strategies

Assay methods and devices that are known in the art can be adapted for the detection and analysis of the biomarkers disclosed herein. In some embodiments, the presence or amount of a biomarker can be determined, for example, using antibodies or other specific binding agents specific for each biomarker and detected through specific binding. Any suitable immunoassay may be utilized, for example, enzyme-linked immunoassays (ELISA), radioimmunoassays (RIAs), competitive binding assays, and the like. Specific immunological binding of the antibody or ligand to the biomarker can be detected directly or indirectly. Direct labels include fluorescent or luminescent tags, metals, dyes, radionuclides, and the like, attached to the antibody. Indirect labels can comprise various enzymes known in the art, such as alkaline phosphatase, horseradish peroxidase and the like.

In general, the analysis of biomarkers can be carried out in a variety of physical formats. For example, the use of microtiter plates or automation can be used to facilitate the processing of large numbers of test samples. Alternatively, single sample formats can be developed to facilitate immediate treatment and diagnosis in a timely fashion, for example, in ambulatory transport or emergency room settings.

The use of immobilized antibodies and/or other binding agent specific for the biomarkers is also contemplated for the detection of the biomarkers described herein. The antibodies could be immobilized onto a variety of solid supports, such as magnetic or chromatographic matrix particles, the surface of an assay place (such as microtiter wells), pieces of a solid substrate material or membrane (such as plastic, nylon, paper), and the like. In some embodiments, an assay strip can be prepared by coating the antibody or a plurality of antibodies in an array on solid support. This strip could then be dipped into the test sample and then processed quickly through washes and detection steps to generate a measurable signal, such as a colored spot. For separate or sequential assay of biomarkers, suitable apparatuses include clinical laboratory analyzers such as the ElecSys (Roche), the AxSym (Abbott), the Access (Beckman), the ADVIA™ or CENTAUR™ (Bayer) immunoassay systems, the NICHOLS ADVANTAGE™ (Nichols Institute) immunoassay system, and the like.

In some embodiments, apparatuses perform simultaneous assays of a plurality of biomarkers using a single test device. Particularly useful physical formats comprise surfaces having a plurality of discrete, addressable locations for the detection of a plurality of different analytes. Such formats include, for example, protein microarrays, or “protein chips” (as described in, for example, Ng et al., J. Cell Mol. Med. 6: 329-340 (2002), incorporated herein by reference) and certain capillary devices (as described in, for example, U.S. Pat. No. 6,019,944, incorporated herein by reference). In these embodiments, each discrete surface location may comprise antibodies to immobilize one or more analyte(s) for a biomarker for detection at each location. The surface locations may comprise one or more discrete particles immobilized at discrete locations of a surface, where the microparticles comprise antibodies or other specific binding agents to immobilize a selected biomarker for detection.

In some embodiments, assay devices can comprise for each assay a first antibody conjugated to a solid phase and a second antibody conjugated to a signal development component for the performance of a sandwich immunoassay. The assay devices for the sandwich assay can comprise a sample application zone and a flow path from the sample application zone to a second device location where the first antibody is conjugated to a solid phase. Additional elements, such as filters to separate plasma or serum from blood, mixing chambers, and the like may be included within a device design Other measurement strategies applicable to the methods described herein include, for example, chromatography (such as HPLC), mass spectrometry, receptor-based assays, and combinations thereof.

A panel consisting of the biomarkers referenced above may be constructed to provide relevant information related to differential diagnosis. Such a panel may be constructed using 1, 2, 3, 4, 5, 10, or more or individual biomarkers. The analysis of a single biomarker or subsets of biomarkers comprising a larger panel of biomarkers could be carried out by one skilled in the art to optimize clinical sensitivity or specificity in various clinical settings. These potential clinical settings include, but are not limited to ambulatory, urgent care, critical care, intensive care, monitoring unit, inpatient, outpatient, physician office, medical clinic, and health screening settings. Furthermore, one skilled in the art can use a single biomarker or a subset of biomarkers selected from a larger panel of biomarkers in combination with an adjustment of the diagnostic threshold in each of the aforementioned settings to optimize clinical sensitivity and specificity. The clinical sensitivity of an assay is defined as the percentage of those with the disease that the assay correctly predicts, and the specificity of an assay is defined as the percentage of those without the disease that the assay correctly predicts. See, Tietz Textbook of Clinical Chemistry, 2nd.sup. edition, Carl Burtis and Edward Ashwood eds., W. B. Saunders and Company, p. 496, incorporated herein by reference.

In another embodiment, a kit can be provided for the analysis of biomarkers. Such a kit can comprise components and/or reagents for the analysis of at least one test sample and/or instructions for performing the assay. Suitable reagents can include, for example, antibodies that detect one or more biomarker of interest (such as desmosine and/or PYR) immobilized on a solid phase, antibodies that detect one or more biomarker of interest (such as desmosine and/or PYR) conjugated to a detectable label, or in further embodiments both solid phase and detectably labeled antibodies. Optionally, the kits may contain one or more instructions for using information obtained from immunoassays performed for a biomarker panel to rule in or out certain diagnoses. Such instructions may be stored on a computer-readable media that provide programming instructions for converting one or more assay signals into a diagnosis or prognosis and/or human-readable material such as labels or package inserts containing instructions that an operator may use to manually convert one or more assay signals into a diagnosis or prognosis.

### Selection of Antibodies and Specific Binding Agents

In some embodiments, antibodies and/or specific binding agents relating to a specific biomarker are used in a particular assay for the biomarker. The generation and selection of antibodies may be accomplished several ways. For example, one way is to purify a peptide of interest or to synthesize a peptide of interest using, e.g., solid phase peptide synthesis methods well known in the art. See, e.g., “Guide to Protein Purification,” Murray P. Deutcher, ed., Meth. Enzymol. Vol 182 (1990) and “Solid Phase Peptide Synthesis,” Greg B. Fields ed., Meth. Enzymol. Vol 289 (1997), both of which are incorporated herein by reference. The selected peptides may then be injected, for example, into chicken, mice or rabbits, to generate polyclonal or monoclonal antibodies, which can be purified from fluids from the animal. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y., incorporated herein by reference. One skilled in the art will also appreciate that binding fragments or Fab fragments which mimic antibodies can also be prepared from genetic information by various procedures. For a further description of antibody manipulation see Antibody Engineering: A Practical Approach (Borrebaeck, C., ed.), 1995, Oxford University Press, Oxford and J. Immunol. 149, 3914-3920 (1992), both of which are incorporated herein by reference.

In addition, numerous publications have reported the use of phage display technology to produce and screen libraries of polypeptides for binding to a selected target, such as an enzyme. See, e.g., Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698, in which all four of these publications are incorporated herein by reference. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between peptides and their genetic material allows simultaneous mass screening of very large numbers of phage bearing different peptides. Phage displaying a peptide with affinity to a target bind to the target, and this phage are enriched by affinity screening to the target. The identity of peptides displayed from the enriched phage can be determined from their respective genomes. Using these methods a peptide identified as having a binding affinity for a desired target can then be synthesized in bulk by conventional means, see, e.g., U.S. Pat. No. 6,057,098, which is incorporated by reference. In alternative or additional embodiments, known substrates for an enzyme or the like can be used to generate specific binding agents. In particular, a selected fragment of a substrate can be tested for affinity with the enzyme.

The antibodies or specific binding agents that are generated by these methods may then be selected by first screening for affinity with the purified polypeptide of interest and, if desired, by comparing the results to the affinity and specificity of the antibodies with polypeptides that are desired to be excluded from binding. For example, the screening procedure can involve immobilization of the purified polypeptides in separate wells of microtiter plates. The solution containing a potential antibody or groups of antibodies can then be placed into the respective microtiter wells and incubated for about 30 min to 2 h. The microtiter wells can then be washed and a labeled secondary antibody (for example, an anti-mouse antibody conjugated to alkaline phosphatase if the raised antibodies are mouse antibodies) is added to the wells and incubated for about 30 min and then washed. Substrate for the alkaline phosphatase can be added to the wells, and a color reaction appears where antibody to the immobilized peptide(s) is present.

The antibodies or specific binding agents selected in this screening process can then be further analyzed for affinity and specificity in the specific assay design. In the development of immunoassays for a target protein or peptide, the purified target protein or peptide can be used as a standard for the evaluation of the sensitivity and specificity of the immunoassay using a candidate antibodies or other specific binding agent. Since the binding affinity of various antibodies may differ and since certain antibody pairs (e.g., in sandwich assays) can interfere with one another with respect to binding, for example, due to steric hindrance, evaluation of antibodies or specific binding agents in actual assay formats can be very helpful with respect to an ultimate selection process.

### Selecting a Treatment Regimen

The ability to detect a risk for aneurysm and/or to monitor the progression of the disease can reduce fatalities from an aneurysm through the detection of asymptomatic aneurysms that can hemorrhage as well as to provide for earlier treatment and monitoring. The development of an aneurysm in a patient can go though several stages in a matter of years. For example, an aorta that has a diameter of 1.7 cm in a healthy individual can increase into 3.0 cm when the individual develop early-stage aneurysm. The early-stage aneurysm can develop into moderate aneurysm where the diameter of the aorta is increased to 4.0 to 5.0 cm. When the aorta in a patient becomes larger than 5.5 cm, the late stage aneurysm is reached and surgical intervention is commonly needed at this point. As discussed earlier, the size of an aneurysm does not always correlate well with the future progress of the disease or the likelihood of aneurysm rupture.

About 85% of all AAA patients are under the categories of early and moderate aneurysm, which are often undetected before the aneurysm reaches a more serious stage. The biomarkers disclosed herein can be used for diagnosis and monitoring of aneurysm at various stages of development as well as for estimating the likelihood of aneurysm rupture. Early and moderate aneurysm can be treated using compositions and devices disclosed in U.S. Patent Application Publication No. 2007/0281026 to Vyavahare et al, entitled “Elastin Stabilization of Connective Tissue”, U.S. Provisional Patent Application Ser. No. 61/066,688 to Isenburg et al. entitled “Treatment of Aneurysm with Application of Elastin Stabilizing Agent Embedded in a Delivery System” and 61/113,881 to Isenburg et al. entitled “Compositions for Tissue Stabilization”, U.S. patent application Ser. No. 12/173,726 to Ogle et al. entitled “Devices for the Treatment of Vascular Aneurysm”, all incorporated herein by reference.

### EXAMPLE

### Detection of Urinary Desmosine in Rats

The detection of urinary desmosine has been correlated with aneurysm development in a rat model. Specifically, increased amounts of desmosine in urine were observed in the rats after conditions for the development of aneurysms was induced.

Urinary desmosine level in rats was measured over the course of 6 weeks. The aneurysm model used was based on the perivascular application of a high concentration calcium chloride (CaCl₂) solution, a method originally used to induce aneurysms in rabbit carotid arteries, as described in Gertz et al., J Clin Invest 1988;81(3):649-656, incorporated herein by reference. This approach has more recently been used on abdominal aorta of rodents. See, e.g., Freestone et al., Arterioscler Thromb Vasc Biol 1995;15(8):1145-1151, Freestone et al., Arterioscler Thromb Vasc Biol 1997;17(1):10-17 and Tambiah et al. Br J Surg 2001;88(7):935-940, all of which are incorporated herein by reference. This model results in a localized mild insult to the arterial tissue. In most studies, a significant increase in aortic diameter (indicative of aneurysm formation) was observed after 3 to 6 weeks post-injury. For this example, the CaCl₂-based chemical injury was performed on rat aorta.

Adult male Sprague-Dawley rats were placed under general anesthesia (2% to 3% isoflurane), allowing for a midline incision along the abdomen in order to expose the infrarenal abdominal aorta. Once exposed, the abdominal aorta was treated with 0.5 M CaCl₂ solution for 15 minutes using a strip (1.5 cm×0.5 cm) of presoaked 8-ply sterile cotton gauze. After treating with CaCl₂, the gauze was removed and the abdominal cavity was closed with subcutaneous suture, followed by surgical staples. The rats were then allowed to recover. Aneurysms progressively form in the rats in a matter of weeks. A total of 10 rats were used.

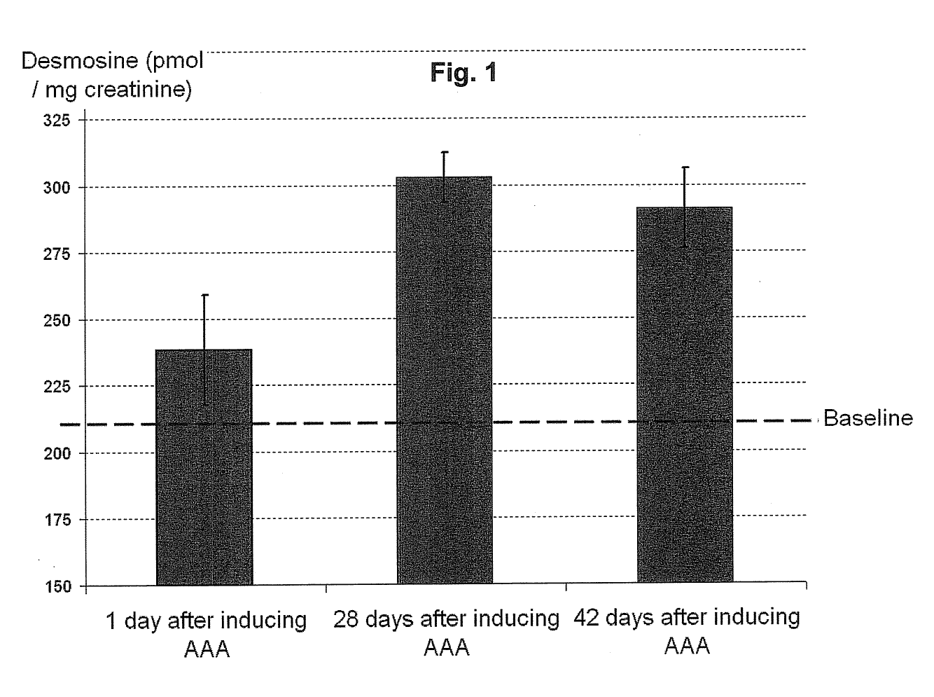

Urine was collected at 1 day, 28 days and 42 days intervals from the rats after surgery and assayed to detect desmosine. As is common with urinalysis methods, the desmosine detected were normalized to creatinine in the sample. An average desmosine value was obtained for the rats from urine collected prior to the aneurysm-inducing surgery to form a baseline desmosine value. The data obtained were presented in FIG. 1. Slightly elevated desmosine level relative to pre-surgery baseline is observed first day after the aneurysm-inducing surgery. It is believed that 3 to 6 weeks post surgery, significant aneurysm is developed in the treated rats. Accordingly, significant increase of desmosine is detected 28 days after the surgery. The level of desmosine at 42 days post-surgery is comparable to that of 28 days post-surgery. The results demonstrated that desmosine was measurable in the urine from rats under conditions where aneurysms are expected to develop, and the data suggests that there is a correlation between progression of aneurysm and an increase in systemic urinary desmosine. Desmosine was quantified by radio-immunoassay (RIA) detailed in Starcher et al., “A role for neutrophil elastase in the progression of solar elastosis,” Connect Tissue Res. 1995; 31:133-140 and Starcher et al., “Measurement of urinary desmosine as an indicator of acute pulmonary disease,” Respiration. 1995; 62(5):252-257, both of which are incorporated herein by reference.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. All patents, patent applications, and publications referenced herein are hereby incorporated by reference herein to the extent that the incorporated material is not contrary to any of the explicit disclosure herein.

We claim:

1. A method for diagnosing an aneurysm, an aneurysm related disorder or an increased propensity thereof, estimating the stage of an existing aneurysm, or determining the propensity of possible future aneurysm rupture in a patient, the method comprising:
correlating levels of a plurality biomarkers measured within a biological sample obtained from a patient with a prescribed formula to evaluate the likelihood of aneurysm, the stage of an exiting aneurysm, or the likelihood of aneurysm rupture,
wherein the plurality of biomarkers comprise at least an elastin degradation product and a collagen degradation product.

2. The method of claim 1 wherein the elastin degradation product is desmosine, isodesmosine, or combination thereof.

3. The method of claim 1 wherein the collagen degradation product is pyridinoline, deoxypyridinoline, or a combination thereof.

4. The method of claim 1 wherein the biological sample is a urine sample, a blood sample, or a combination thereof.

5. The method of claim 1 wherein the formula comprises the comparison of a level of the biomarkers against a reference level of the biomarkers, wherein the reference levels have been obtained through the measurement of the level of the plurality of biomarkers detected from healthy individuals or control patients at predetermined stage of aneurysm.

6. The method of claim 1 wherein the formula comprises the comparison of a level of the biomarkers against a reference level of the biomarkers, wherein the reference levels are obtained based on previously obtained values from the patient.

7. The method of claim 1 wherein correlation of the plurality of biomarker levels is repeated in an interval from about six months to about 5 years, and wherein at least one repeated biomarker level is compared with the previously obtained value from the patient.

8. The method of claim 7 wherein the interval is from about 10 months to about 18 months.

9. The method of claim 1 wherein the plurality of biomarkers further comprises matrix metalloproteinase.

10. The method of claim 9 wherein the matrix metalloproteinase comprises matrix metalloproteinase 1, 2, 8, 9, 12, 13, 18, or a combination thereof.

11. The method of claim 9 wherein the elastin degradation product comprises desmosine, isodesmosine, or a combination thereof, the collagen degradation product comprises pyridinoline, deoxypyridinoline, or a combination thereof, and the matrix metalloproteinase comprises matrix metalloproteinase 1, 2, 8, 9, 12, 13, 18, or a combination thereof.

12. The method of claim 1 wherein the plurality of biomarkers further comprises elastin degradation peptide, procollagen-IIIN-terminal propeptides, N-telopeptides of type I collagen, tissue inhibitors of metalloproteinases, tissue plasminogen activator, urokinase plasminogen activator, homocysteine or a combination thereof.

13. A method for diagnosing an aneurysm, aneurysm related disorder or an increased propensity thereof, estimating the stage of an existing aneurysm, or determining the propensity of possible future aneurysm rupture in a patient, the method comprising:
correlating levels of a plurality of biomarkers measured within a biological sample obtained from a patient with a prescribed formula to evaluate the likelihood of aneurysm, the stage of an existing aneurysm, or the likelihood of aneurysm rupture
wherein the plurality of biomarkers comprise at least desmosine, isodesmosine, and a metalloproteinase.

14. The method of claim 13 wherein the matrix metalloproteinase comprises matrix metalloproteinase 1, 2, 8, 9, 12, 13, 18, or a combination thereof.

15. The method of claim 13 wherein the matrix metalloproteinase comprises matrix metalloproteinase 2, 9, or a combination thereof.

16. The method of claim 13 wherein the sample is a urine sample, a blood sample, or a combination thereof.

17. The method claim 13 wherein the formula comprises the comparison of a level of the biomarkers against a reference level of the biomarkers, wherein the reference levels have been obtained from healthy individuals or control patients at pre-determined stage of aneurysm.

18. The method of claim 13 wherein the formula comprises he comparison of a level of the biomarkers against a reference level of the biomarkers, wherein the reference levels are obtained based on previously obtained values from the patient.

19. The method of claim 13 wherein the correlation of the levels of biomarkers is repeated in an interval from about six months to about 5 years.

20. The method of claim 13 wherein the plurality of biomarkers further comprises a collagen degradation product.

21. The method of claim 20 wherein the collagen degradation product comprises pyridinoline, deoxypyridinoline, or a combination thereof.

22. The method of claim 13 wherein the plurality of biomarkers further comprises elastin degradation peptide, procollagen-IIIN-terminal propeptides, N-telopeptides of type I collagen, tissue inhibitors of metalloproteinases, tissue plasminogen activator, urokinase plasminogen activator, homocysteine or a combination thereof.

23. A kit for performing a test to diagnose an aneurysm, an aneurysm related disorder or an increased propensity thereof, estimate the stage of an existing aneurysm, or determine the propensity of possible future aneurysm rupture in a patient, the kit comprising:
one or more agents for detecting the level of each of a plurality of biomarkers in a biological sample from a patient to determine the levels of the plurality of biomarkers in the sample,
wherein the biomarkers are selected such that a correlation of the levels of the biomarkers with respect to known distributions of biomarker levels in a population can be used as an estimate of the likelihood of aneurysm, the stage of an existing aneurysm, or the likelihood of aneurysm rupture in the patient.

24. The kit of claim 23 wherein at least one agent can be used for the evaluation of the level of desmosine or isodesmosine in the sample.
